(19) United States
(12) Patent Application Publication
(10) Pub. No.: US 2003/0183231 A1
(43) Pub. Date: Oct. 2, 2003

(54) Apparatus & method for determining at least one parameter of a respiratory system's (RS) mechanical properties

(76) Inventors: Giuseppe Pedulla, Brampton (CA); Rod MacDonald, Guelph (CA)

Correspondence Address: MEREK, BLACKMON & VOORHEES, LLC, 673 South Washington Street, Alexandria, VA, 22314, US

(21) Appl. No.: 10/359,246

(22) Filed: Feb. 6, 2003

Related U.S. Application Data
(60) Provisional application No. 60/354,954, filed on Feb. 11, 2002.

(51) Int. Cl. A61M 15/00

(52) U.S. Cl. 128/204.18

(57) ABSTRACT

A device is provided which measures at least one mechanical property of the Respiratory System (RS) using an input perturbation which linearises the RS and measures the resulting output waveform. In its' full embodiment this device provides for a complete characterization of the RS′ mechanical state. One preferred embodiment utilizes appropriate transducers and associated processing to produce a plot that is able to locate lower inflection point (LIP), maximal slope (MS), over distension (O), upper inflection point (UIP), and closing volume (CV) in a lung with far greater sensitivity and accuracy than the ‘gold standard’ pressure volume curve. Supporting electronics and software provide for, at least, a display of trends, a xy display, and a display of spectral content. An integral alarm alerts the clinician to low signal conditions that may indicate, for example, a loss of respiratory effort. All devices in this family rely on one easily satisfied assumption. Namely that the input perturbation be an appropriately short duration pulse which is sent into the RS. Each embodiment requires only a slight modification in configuration. Namely different bandwidths are used to obtain the signal components of interest and one or more channels of acceleration are utilized. As such it is reasonable to define the entire set of embodiments as “one invention”.

This application claims priority from provisional patent application 60/354,954, files Feb. 11, 2002.

### FIELD OF THE INVENTION

This invention relates to measuring RS mechanical properties of living organisms including at least humans.

### BACKGROUND OF THE INVENTION

Mechanical ventilation, the most frequent type of intervention administered to patients in respiratory failure, provides life-saving respiratory support. At the same time this intervention leads to ventilator-associated lung injury (VALI). VALI has received international attention because of its' associated increase in mortality. In short VALI tends to create populations of abnormal lung units that are unstable and collapse easily. Further some of these units become unrecruitable and cannot be reopened.

Three mechanisms contribute to VALI associated pathology: over distension injury, shearing injury and oxygen toxicity. These three ultimately result in an injurious mechanism known as biotrauma.

Overdistension injury results when excessive volume is delivered to areas of the lung resulting in that area exceeding its' local maximum volume capacity. This can result from high pressure or high volume ventilation. Furthermore unequal distribution of delivered gas to the lung can lead to overdistension injury in healthy lung units without achieving ventilation to injured lung units.

Shearing injury refers to microscopic damage caused by repetitive opening and closing of terminal lung units. This injury is characteristic of conventional high volume/high-pressure ventilation.

Oxygen toxicity results when lung tissue is exposed to high oxygen partial pressure levels for as little as 20 minutesf. This damage leads to fibrotic changes in lung tissue, which in turn further decrease compliance and diffusion capacity.

A final common pathway for these three mechanisms is biotrauma. In biotrauma there is a local release of inflammatory mediators, which in turn perpetuate the cascade of lung inflammation and worsen lung injury. Furthermore these mediators are released into the blood stream resulting in damage to other organ systems. In a recent trial it was found that these mediators were attenuated by a ventilation strategy that minimized overdistension and cyclic alveolar collapse.

In addition the cumulative effects of these injuries are known to lead to decreased lung compliance, increased pulmonary shunt with progressive hypoxemia, and decreased alveolar volume available for Carbon Dioxide (CO2) clearance and Oxygen (O2) uptake. Consequently, early detection of the factors, which lead to VALI, is an area of extreme interest.

In a second related area other lung diseases are associated with uneven gas distribution in the lung. For example, high airway resistance and ‘gas trapping’ characterize Asthma. Another disease, C.O.P.D. is characterized by areas of overdistension and a decrease in alveolar surface area. In newborn infants, extremely high work of breathing, large areas of collapsed lung units, and poor gas distribution characterize some respiratory diseases.

A third related area is associated with ventilator-patient asynchrony. This problem is caused by the fact that there is a delay between a patient's effort to breathe and a ventilator's response. This leads to significant phase delays between flow delivery and patient requirements. In short this can mean increased oxygen requirements, prolonged weaning, further worsening of lung disease, and the need for increasingly drastic interventions.

Finally, another related area can be found in sleep medicine. Two of the many physiological parameters measured during a sleep study are chest and abdominal movements. These are accomplished by the use of ‘bands’ that encircle the patient's body. These bands are considered problematic and require constant monitoring by the Sleep Technologist. Indicators used to measure the effectiveness of current interventions are limited. Although currently monitored parameters (such as arterial blood gases and O2sats) provide an idea of biochemical effectiveness they do not provide information regarding the mechanical effectiveness of ventilation. Gas dynamic parameters such as pressure, flow, and volume are ambiguous at best. Their interpretation requires a series of questionable assumptions. Even under the best of conditions these measurements are difficult to perform and full of potential sources of error. Reliable techniques that can characterize mechanical properties of the RS are few and far between.

There are currently several approaches aimed at determining RS mechanical properties. A common feature of these approaches is the need to measure one or more of pressure, flow and volume. Typically one of these variables is used as an independent variable and the other two are then measured. Of all the techniques available the Pressure Volume (PV) Curve is considered the ‘gold standard’ against which others are measured. In this method the relationship between pressure and volume is plotted on a graph. Traditionally volume (usually the independent variable) is plotted on the y-axis and pressure is plotted on the x-axis. Several points of interest are then identified. These points are:

1) Lower Inflection Point (LIP): The point on inflation at which volume begins rising at a faster rate when compared to equal increases in pressure.

2) Maximal Slope (MaxS): The point where the slope is greatest on inflation.

3) Over Distension (OD): The point on inflation where the curve ‘flattens’ out suggesting that volume increases very little for any given increment in pressure.

4) Upper Inflexion Point (UIP): The point on the deflation curve where volume begins dropping at a faster rate for any given change in pressure.

5) Closing Volume (CV): The point on the deflation curve where volume decreases at a great rate towards zero.

Clinically it is difficult to construct a repeatable PV curve and the process is highly interventional.

Reviews of current methods disclose several serious sources of potential artifacts. As a summary some of these are:

1. It is assumed that the change in volume (ΔV) is comprised of only “lung volume” whereas in fact some of the volume must be lost as compressible volume within the lung and associated tubing and does not contribute to the actual volume delivered to the lung. Furthermore, if the lung tissue's compliance is high then the measured compliance will appear to be high but in fact a lot of volume is lost in distending smaller bronchi and alveolar ducts so alveolar ventilation can be quite low. Consequently the measure of volume, which is frequently used as an independent variable, can be in error.

2. There is no way to separate lung compliance from the effects of chest wall and abdomen. Furthermore, any measurements obtained can only represent global averages of a system that is known to display regional variations. Careful studies by Gattinonni have shown that volume is preferentially delivered to good lung units. Consequently these methods can only measure ventilation to the good lung. These methods can only indicate over distension of good units without any reference to what is happening with the diseased units. Furthermore none of the methods can claim spatial resolution and therefore cannot locate regional differences.

3. In cases of elevated airway resistance and uneven time constants, pressure and flow may never plateau thereby leading to errors introduced by non-linear flow dynamics.

4. During the time that the thorax is being inflated with a given volume several events occur including O2consumption and CO2production. These effects can account for about 17% error in measured volumes.

5. Some methods require paralysis and breath holds. Consequent haemodynamic and temperature changes may make it difficult if not impossible to achieve a steady state condition.

6. Gas introduced from a super syringe changes conditions from ambient to body temperature and pressure. This can account for an additional 12% error.

7. Regardless of how carefully the procedure is performed inflection points can be very hard to see and are almost always ‘eyeballed’ by clinicians introducing interpretive error.

8. Most of the methods are based on lumped parameter linear lung models. However it is acknowledged that the lung is highly non-linear over the range of bulk convective volume displacements used by classical methods. Furthermore the lung is not time invariant and posses memory. Finally it is unlikely that a lumped parameter can accurately model respiratory disease because it is characterized by non-homogeneous changes in material properties at all anatomical levels.

Most techniques rely on the measurement of flow, pressure and volume and therefore are subject to at least some of the errors listed above.

### SUMMARY OF THE INVENTION

The invention therefore provides a device, which solves the problems discussed above.

A careful analysis of this problem clearly suggests the need for a different point of view. Since most of the aforementioned problems involve a ‘mechanical pathology’ it makes sense to concentrate efforts at measuring the mechanical state of the RS. We must answer questions such as, but not limited to, what is the best bias point for lung volume, what is the most mechanically efficient method for achieving ventilation without initiating the VALI cascade, and where are we on the lung's characteristic curve?

The invention is fundamentally different because it directly addresses the need to measure the mechanical state of the RS. The invention measures the output response of a linearised system to an input perturbation at the level of the body surface. It then processes the data so obtained into clinically relevant information.

Accordingly in one of its' aspects the invention provides for a system that determines the mechanical properties of the human lung and provides clinical information indicating, at least, LIP, MaxS, OD, UIP and CV points. Furthermore, the invention provides additional information which, when taken as a whole, can completely characterize the mechanical state of the RS under clinically relevant conditions. For example the invention can provide a measure of Displacement (D) vs. Pressure, Velocity (V) vs. Pressure, and Acceleration (A) vs. Pressure. Furthermore the invention can provide for a measure of the trends in D, V, P and A. In addition the invention can provide real time waveforms of D, V, P and A. Finally the invention can measure Dynamic Mass, Mechanical Impedance, Dynamic Stiffness, Compliance, Mobility, and Accelerance. These examples are merely illustrative but not exhaustive. All this is accomplished by assuring that only one assumption be satisfied. Namely that the perturbation be of such character so as to represent a mere perturbation about the system's bias point. This assumption is satisfied ‘by design’ and therefore the methodology used is robust and unaffected by any uncontrollable factors.

In a further aspect the invention provides the ability to differentiate regional differences in the RS's mechanical state. This property can be termed Spatial Resolution (SR). SR allows the clinician the ability to determine regional pathology thus identifying, at least, areas of overdistension. This feature addresses the concerns with uneven distribution of gas during ventilation of the RS.

In a further aspect the invention allows the ability to track phase relationships between input flow/pressure and output response. This property can be termed Temporal Resolution (TR). TR allows for, at least, a new triggering system for ventilators that displays enhanced sensitivity to patient effort. This feature addresses concerns with work of breathing and ventilator asynchrony.

In yet a further aspect the invention allows the ability to track body surface movement. This feature can be deployed to, at least, sleep labs in order to monitor chest wall and abdominal movements.

Further advantages of the invention will become apparent from the following description taken together with the accompanying drawings.

### BRIEF DESCRIPTION OF THE DRAWINGS

For a better understanding of the present invention, and to show more clearly how it may be carried into effect, reference will now be made, by way of example, to the accompanying drawings which show the preferred embodiments of the present invention in which:

FIG. 1 is an illustration of a device that can be used for delivering an input perturbation into the RS. It is possible and equally valid to replace this signal with that generated by another device such as but not limited to a high frequency oscillator or any suitable device with the capability of generating a short duration pulse of pressure. The pulse so introduced acts to cause a local dilation in the lung's system of tubes as well as a local dilation of volume in the alveolar sacs. This can be pictured as a ripple traveling through the respiratory system and finally exiting it.

FIG. 2 is an illustration of sensor placement for various embodiments of the device. Other configurations are possible and this figure is merely an illustration of some possibilities and should be considered as illustrative and not restrictive.

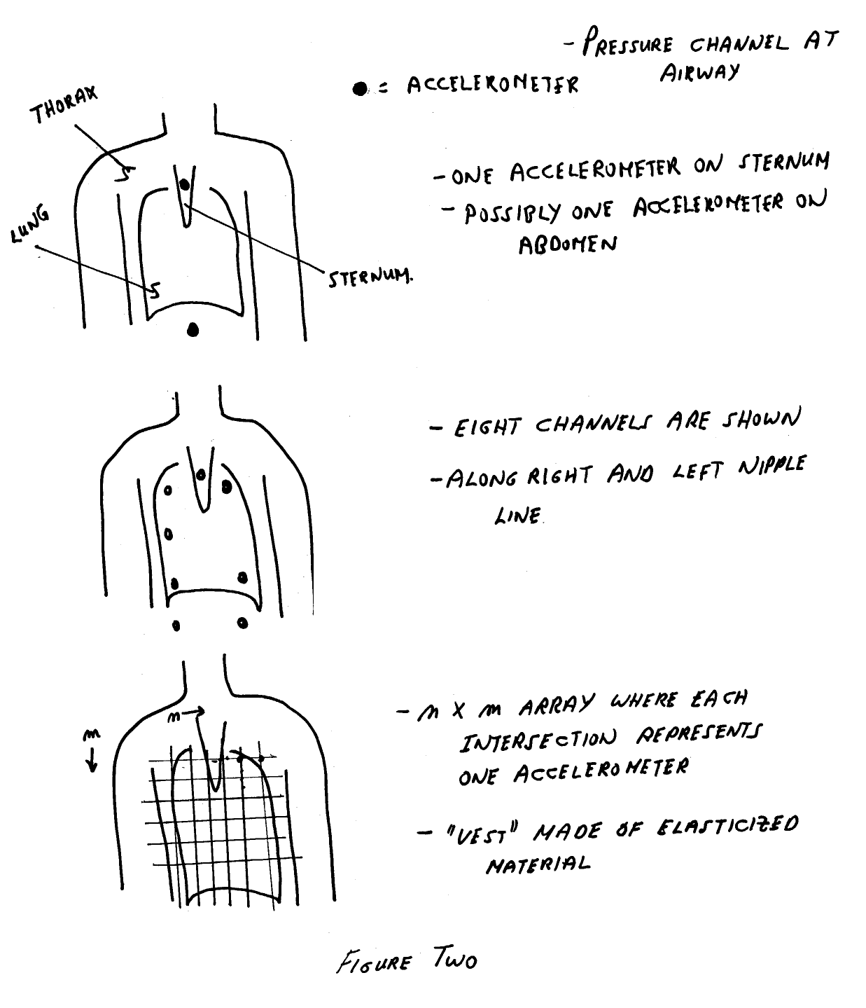

FIG. 3 is a detail of a typical sensor and cable construction for one sensor used by the invention.

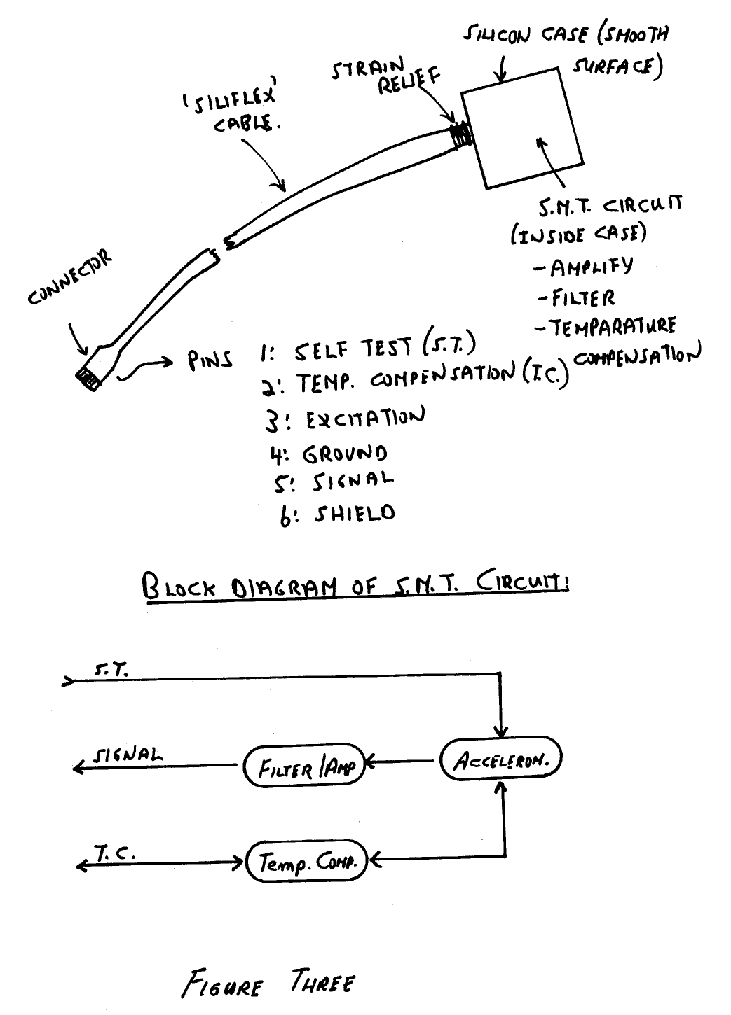

FIG. 4 illustrates one possible method of firmly affixing the sensor to a body surface.

FIG. 5 is a block diagram of the preferred embodiment illustrating the required subsystems necessary for operation.

FIG. 6 is a block diagram of a software embodiment that can be deployed quickly for use in, for example, research labs.

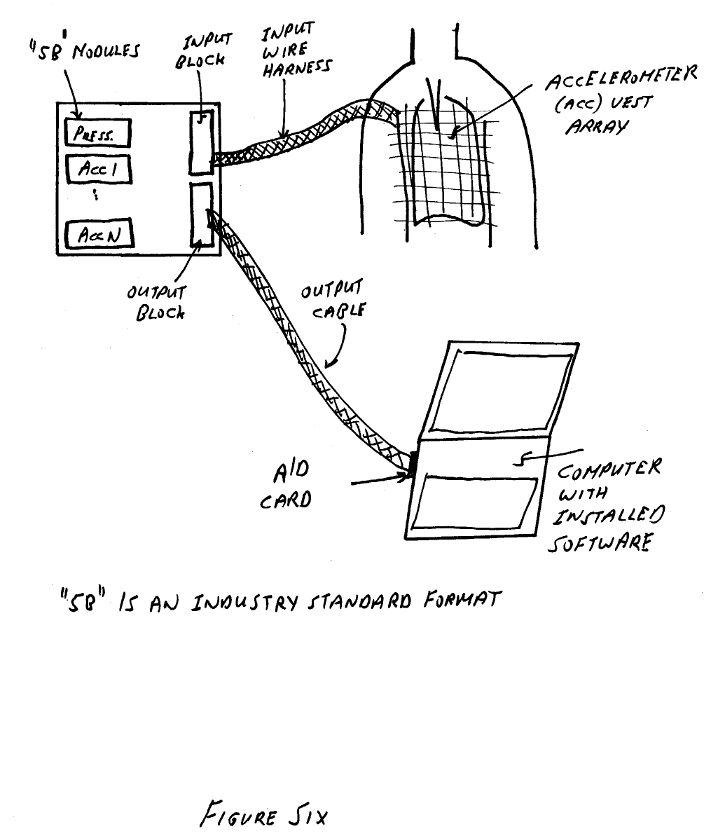

FIG. 7 is a sample display from one embodiment illustrating the type of data obtained by processing the input stream.

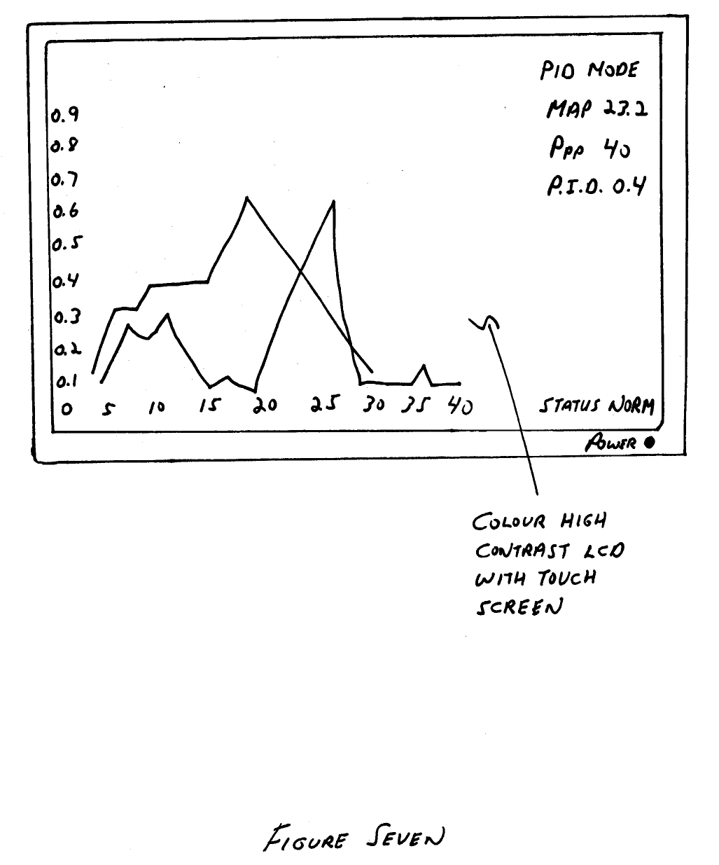

### DESCRIPTION OF THE PREFERRED EMBODIMENT

The present invention may be embodied in a number of different forms. However, the specification and drawings that follow describe and disclose only some of the specific forms of the invention and are not intended to limit the scope of the invention as defined in the claims that follow herein.

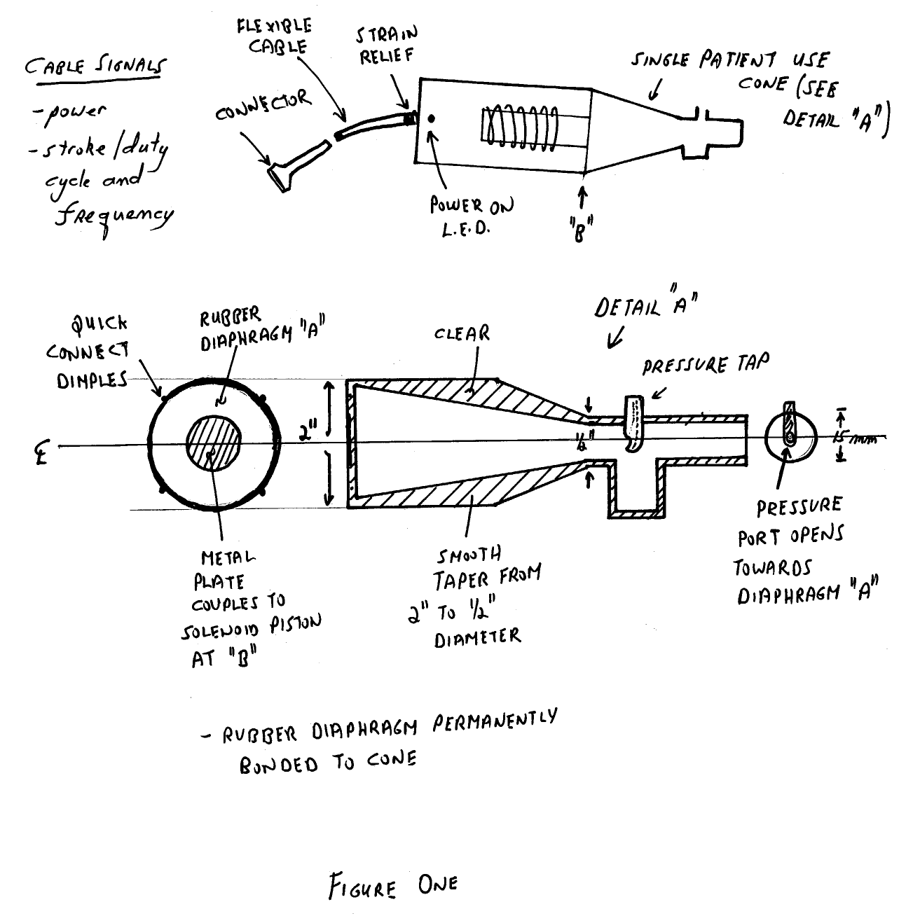

With reference to FIG. 1 a signal from the perturbation device is sent into the RS via, possibly but not limited to, an Endotracheal Tube. It is possible and equally valid to replace this signal with that generated by a high frequency oscillator or one generated by any suitable device with the capability of generating a short duration pulse of pressure. The parameters of this perturbation can be adjusted ‘at the source’ by altering the associated controls. In the case where the PBD is used the preferred embodiment will include a menu choice whereby the user can alter the drive current to the solenoid as well as, at least, the duty cycle of the current. From the user's point of view these controls would be labeled as ‘Amplitude’ and ‘Itime %’ respectively. Other controls could be implemented as needed. This would allow the user to discover relationships we have identified between input perturbation and output response. In one embodiment, the PBD could be used, for example, to deliver pulses of appropriate parameters which would be superimposed upon a conventional ventilator breath and which would provide data streams for processing. In one embodiment the PBD must deliver a pulse having the characteristic of being of a short duration and of a small amplitude such that the Transpulmonary pressure swings so induced do not exceed 25% of the peak values normally felt by the RS under specified conditions. In the embodiment shown the PBD is firmly coupled to a patient wye by means of an adapter. This single patient use adapter is gas tight and functions to amplify and direct the perturbation into the patient's endotracheal tube. The adapter is designed for ‘single patient use’ and not intended for reuse. Other embodiments may require other means of coupling the signal into the RS. In some applications this arrangement would, possibly, improve oxygenation by creating an enhancement of diffusive processes within the human lung. However it should be noted that the PBD is not intended to provide ventilation of the RS but merely to provide the perturbing signal required by the invention.

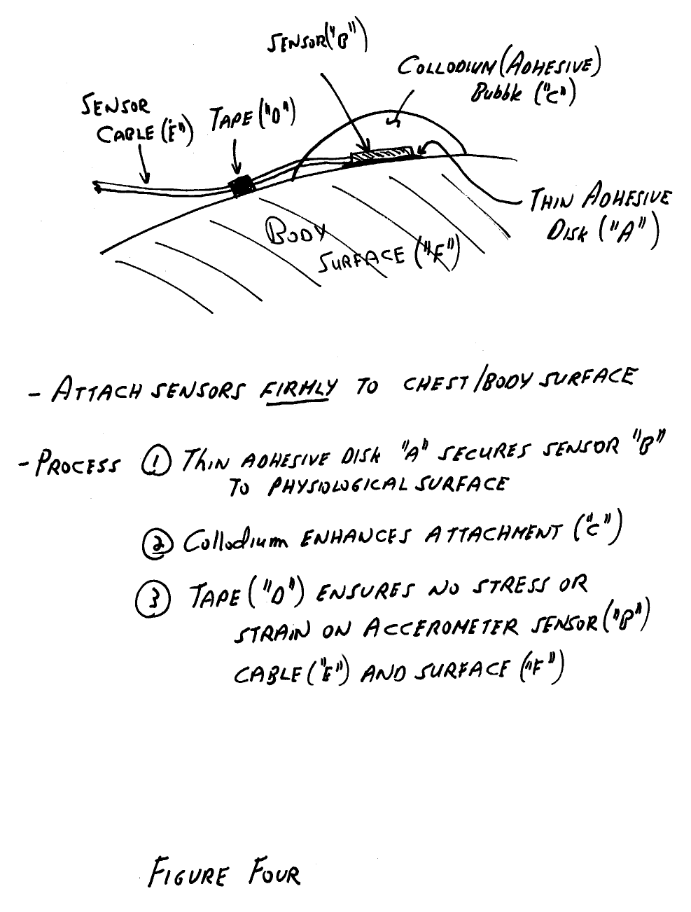

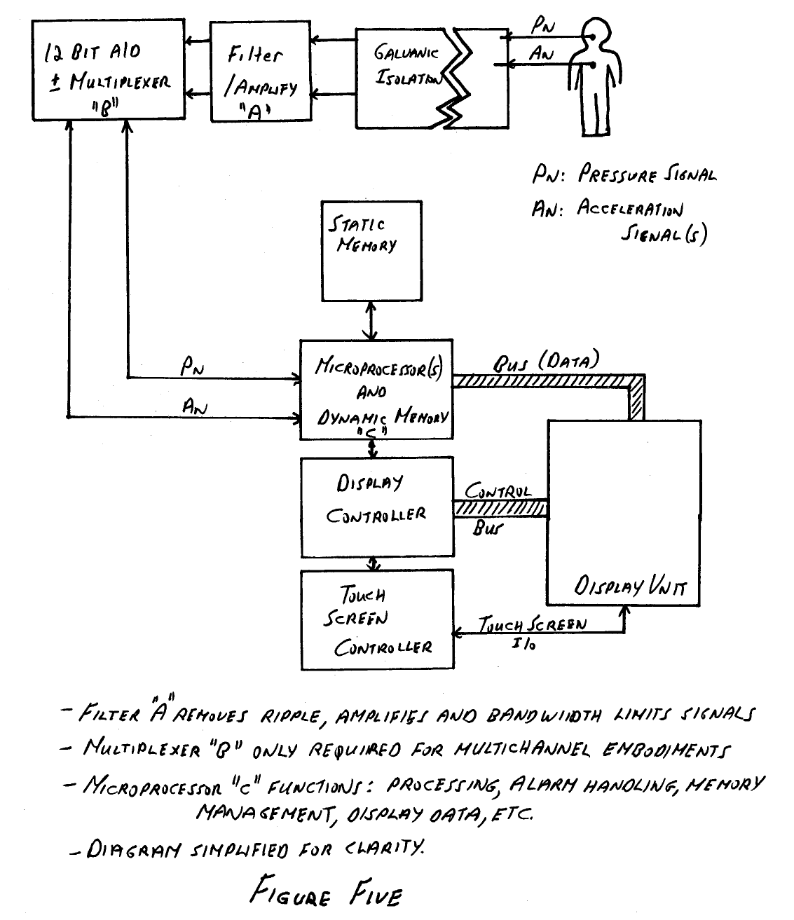

With reference to figures two and three it can be seen that the invention relies on a least two channels of data—pressure and acceleration. It is entirely possible to utilize more than one accelerometer channel up to a plurality of accelerometers, which would thereby constitute an array of accelerometers. Pressure is monitored using a micro machined differential pressure transducer at the input to the RS. Acceleration is monitored using an accelerometer packaged into a single patient use cable with at least one sensor at the mid sternal point of the chest wall and possibly elsewhere on the external surface of the human body. In all embodiments the arrangement(s) of accelerometers must be made with reference to mechanical behaviors and couplings between chest wall, lung, and abdominal components. It is important to affix the sensor firmly to the body surface. One possible method of affixing the accelerometer to the body surface is shown in FIG. 4. The accelerometer signal is pre-filtered and amplified using parameters suitable to the specific embodiment. Acceleration is BW limited to components below around 150 Hz. Depending on the embodiment filtering parameters may be varied with a lower bound of 0 Hz and an upper bound of 150 Hz. Depending on the embodiment filters may be of the low pass, high pass, band pass or notch type. In the embodiment illustrated by FIG. 5 these parameters would be, at least, a high frequency cutoff of 150 Hz.

With reference to FIG. 5 the accelerometer is then isolated from the patient using three-port isolation and its' signal is fed into signal conditioning circuitry. Signal conditioning is performed on all channels to buffer, amplify and further filter the signals. Depending on the embodiment these additional filters may be of the low pass, high pass, band pass or notch type. These signals are then digitized at a rate of at least 4 times the highest post-filtered frequency component. Signal streams are partitioned into ‘pulses’ by triggering off the pressure channel. The user can define trigger levels based on pressure levels, time, and/or phase relationships between pressure and one or more accelerometer channels. This and further processing may be performed by firmware or software depending on the embodiment. In the preferred embodiment shown digital streams are then sent to memory for storage and display subsystems for display. Several display modes are available. It is possible to display many parameters, for example, in terms of trends (i.e. vs. time) or in terms of each other (i.e. acceleration vs. pressure) or in terms of spectral content (i.e. A vs. frequency). For example a trend display shows values of maximum acceleration over a user selectable time period. The user may select from 1 minute to several hours. Other modes plot data that has been derived via processing of the raw acceleration and pressure data. For example one plot displays maximal displacement vs. mean pressure. If a sufficient number of channels are used the data stream can be mapped to a gray scale plot of a user selectable parameter overlaid onto a diagram of a typical human lung. If a sufficient number of accelerometer channels are used it is possible to develop a 3 dimensional plot of displacement over the entire body surface. A rear panel RS232C port allows downloading of serial data to an external device. In the embodiment illustrated there is a rear panel connection to provide power and control signals to the PBD. The man machine interface is via a touch screen interface. The firmware embodiment provides for upgradeable software via a rear panel port. The software embodiment can be deployed onto a computer that has an A/D card capable of digitizing at a rate of a least 600 Hz per channel and supporting hardware such as, but not limited, to a terminal block.

It is to be understood that what has been described as the preferred embodiments of the invention and that it may be possible to make variations of these embodiments while staying within the broad scope of the invention. Some of these variations have been discussed while others will be readily apparent to those skilled in the art.

The inventors claim:

1. The apparatus and method for determining at least one parameter of a Respiratory System's mechanical properties as described herein and shown in the attached drawings.
